(19) United States
(12) Patent Application Publication
(10) Pub. No.: US 2021/0386374 A1
(43) Pub. Date: Dec. 16, 2021

(54) Animal Physiological Device

(71) Applicant: Nitto Denko Corporation, Osaka (JP)

(72) Inventors: Md Irwan Bin Md Kassim, Singapore (SG); Mohamad Sulhede Bin Samsudin, Singapore (SG); Ananya Chaithanaboon, Bangkok (TH); Usanee Apijuntarangoon, Bangkok (TH); Visit Thaveeprungsriporn, Singapore (SG)

(73) Assignee: Nitto Denko Corporation, Osaka (JP)

(21) Appl. No.: 17/289,118

(22) PCT Filed: Nov. 8, 2019
(86) PCT No.: PCT/SG2019/050548

§ 371 Date: Apr. 27, 2021

(30) Foreign Application Priority Data
Nov. 8, 2018 (SG) 10201809940S

(51) Int. Cl. A61B 5/00; A61B 5/024; A61B 5/021; A61B 5/0205

(52) U.S. Cl. CPC A61B 5/6833; A61B 5/02416; A61B 5/021; A61B 2503/40; A61B 5/6838; A61B 2562/0238; A61B 5/02055

(57) ABSTRACT

The present disclosure generally relates to a physiological device (100) for an animal The physiological device (100) comprises: a housing (120) comprising a channel (122) therethrough; an attachment layer (140) disposed on the housing (120) for attaching the physiological device (100) to an integument portion (50) of the animal; and a sensor unit (160) comprising a set of physiological sensors (162) for measuring physiological signals from the animal integument portion (50), the sensor unit (160) engageable with the channel (122) for axial displacement within the channel (122), wherein when the device (100) is attached to the animal integument portion (50), the sensor unit (160) is axially displaceable within the channel (122) for adjusting contact with the animal integument portion (50) for measuring the physiological signals.

### CROSS REFERENCE TO RELATED APPLICATION(S)

The present disclosure claims the benefit of Singapore Patent Application No. 10201809940S filed on 8 Nov. 2018, which is incorporated in its entirety by reference herein.

### TECHNICAL FIELD

The present disclosure generally relates to an animal physiological device. More particularly, the present disclosure describes various embodiments of a physiological device for an animal and a system for monitoring physiological conditions of animals having the physiological devices attached thereto.

### BACKGROUND

It is evidently known that understanding of physiological conditions of animals may give farmers or researchers useful knowledge and insights about the animals, such as about their health, functions, and states of mind. Monitoring of the animal physiological conditions may provide knowledge on whether the animal has any animal-associated diseases, such as bovine respiratory disease which affects beef cattle. The animal physiological conditions can be monitored by obtaining physiological data from the animals using devices attached on the animals' skin. An example of such device is described in U.S. Pat. No. 10,349,632. This device has a housing attachable to an animal and a sensor assembly disposed within an internal cavity of the housing. However, even though the housing may be attached to the animal, the sensor assembly may not be properly positioned directly to animal skin to measure useful physiological data from the animals.

Therefore, in order to address or alleviate at least one of the aforementioned problems and/or disadvantages, there is a need to provide an improved animal physiological device.

### SUMMARY

According to a first aspect of the present disclosure, there is a physiological device for an animal. The device comprises: a housing comprising a channel therethrough; an attachment layer disposed on the housing for attaching the device to an integument portion of the animal; and a sensor unit comprising a set of physiological sensors for measuring physiological signals from the animal integument portion, the sensor unit engageable with the channel for axial displacement within the channel, wherein when the device is attached to the animal integument portion, the sensor unit is axially displaceable within the channel for adjusting contact with the animal integument portion for measuring the physiological signals.

According to a second aspect of the present disclosure, there is a system for monitoring physiological conditions of an animal. The system comprises a set of physiological devices attachable to the animal, each device comprising: a housing comprising channel therethrough; an attachment layer disposed on the housing for attaching the device to an integument portion of the respective animal; and a sensor unit comprising a set of physiological sensors for measuring physiological signals from the animal integument portion, the sensor unit engageable with the channel for axial displacement within the channel, wherein when the device is attached to the animal integument portion, the sensor unit is axially displaceable within the channel for adjusting contact with the animal integument portion for measuring the physiological signals. The system further comprises an electronic device communicative with the physiological devices for processing the physiological signals to thereby monitor the physiological conditions of the animals.

An animal physiological device according to the present disclosure are thus disclosed herein. Various features, aspects, and advantages of the present disclosure will become more apparent from the following detailed description of the embodiments of the present disclosure, by way of non-limiting examples only, along with the accompanying drawings.

### BRIEF DESCRIPTION OF THE DRAWINGS

FIG. 1A to FIG. 1C are illustrations of various cross-sectional views of a physiological device for an animal, the device having a screw mechanism, in accordance with some embodiments of the present disclosure.

FIG. 2A and FIG. 2B are illustrations of various cross-sectional views of a physiological device for an animal, the device having a clip mechanism, in accordance with some embodiments of the present disclosure.

FIG. 3A to FIG. 3C are illustrations of various external views of the physiological device for the animal having the screw mechanism, in accordance with some embodiments of the present disclosure.

FIG. 4A to FIG. 4C are illustrations of various external views of the physiological device for the animal having the clip mechanism, in accordance with some embodiments of the present disclosure.

FIG. 5 is a schematic illustration of a system for monitoring physiological conditions of an animal using a set of physiological devices for the animal, in accordance with some embodiments of the present disclosure.

FIG. 6A and FIG. 6B are illustrations of physiological signals measured from the animal, in accordance with some embodiments of the present disclosure.

### DETAILED DESCRIPTION

For purposes of brevity and clarity, descriptions of embodiments of the present disclosure are directed to an animal physiological device in accordance with the drawings. While aspects of the present disclosure will be described in conjunction with the embodiments provided herein, it will be understood that they are not intended to limit the present disclosure to these embodiments. On the contrary, the present disclosure is intended to cover alternatives, modifications and equivalents to the embodiments described herein, which are included within the scope of the present disclosure as defined by the appended claims. Furthermore, in the following detailed description, specific details are set forth in order to provide a thorough understanding of the present disclosure. However, it will be recognized by an individual having ordinary skill in the art, i.e. a skilled person, that the present disclosure may be practiced without specific details, and/or with multiple details arising from combinations of aspects of particular embodiments. In a number of instances, well-known systems, methods, procedures, and components have not been described in detail so as to not unnecessarily obscure aspects of the embodiments of the present disclosure.

In the present disclosure, depiction of a given element or consideration or use of a particular element number in a particular figure or a reference thereto in corresponding descriptive material can encompass the same, an equivalent, or an analogous element or element number identified in another figure or descriptive material associated therewith.

References to “an embodiment/example”, “another embodiment/example”, “some embodiments/examples”, “some other embodiments/examples”, and so on, indicate that the embodiment(s)/example(s) so described may include a particular feature, structure, characteristic, property, element, or limitation, but that not every embodiment/example necessarily includes that particular feature, structure, characteristic, property, element or limitation. Furthermore, repeated use of the phrase “in an embodiment/example” or “in another embodiment/example” does not necessarily refer to the same embodiment/example.

The terms “comprising”, “including”, “having”, and the like do not exclude the presence of other features/elements/steps than those listed in an embodiment. Recitation of certain features/elements/steps in mutually different embodiments does not indicate that a combination of these features/elements/steps cannot be used in an embodiment.

As used herein, the terms “a” and “an” are defined as one or more than one. The use of “/” in a figure or associated text is understood to mean “and/or” unless otherwise indicated. The term “set” is defined as a non-empty finite organization of elements that mathematically exhibits a cardinality of at least one (e.g. a set as defined herein can correspond to a unit, singlet, or single-element set, or a multiple-element set), in accordance with known mathematical definitions. The recitation of a particular numerical value or value range herein is understood to include or be a recitation of an approximate numerical value or value range. As used herein, the terms “first” and “second” are used merely as labels or identifiers and are not intended to impose numerical requirements on their associated terms. As used herein, the term “each other” represents a reciprocal relation between two or more elements.

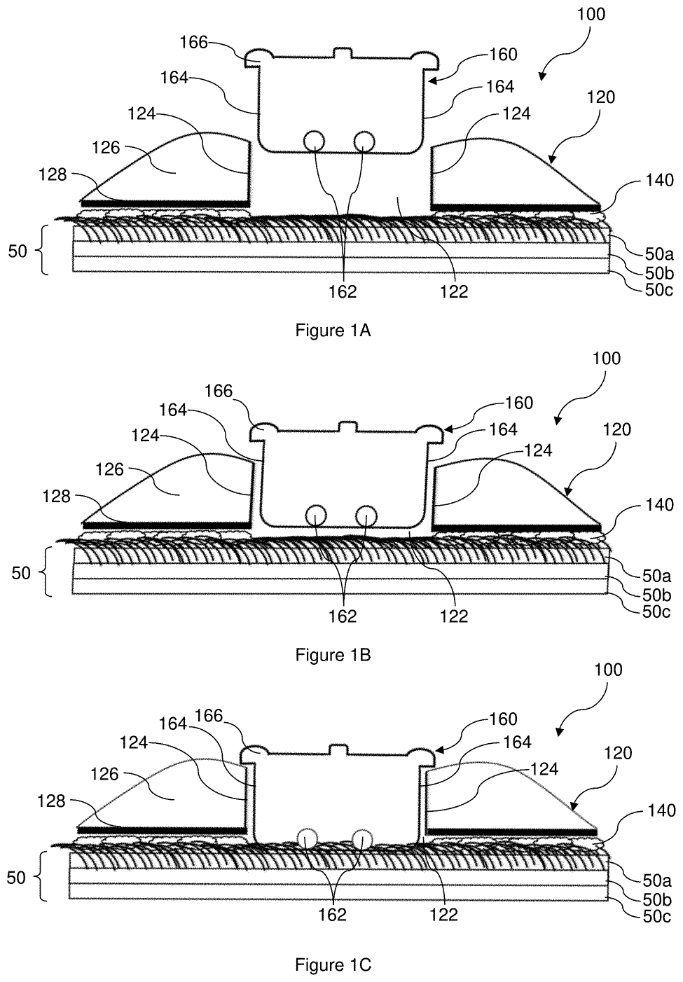

In representative or exemplary embodiments of the present disclosure, there is a physiological device 100 for an animal as illustrated in FIG. 1A to FIG. 1C. The animal may be a dairy cow, beef cow, cattle, buffalo, sheep, goat, pig, horse, dog, and the like. The physiological device 100 includes a housing 120, an attachment layer 140, and a sensor unit 160. The housing 120 includes a channel 122 therethrough. The channel 122 is a holed portion formed through the housing 120, preferably at a central region of the housing 120. The housing 120 and the channel 122 may be of various shapes, such as but not limited to circular, square, rectangular, and elliptical.

The attachment layer 140 is disposed on the housing 120 for attaching the device 100 to an integument portion 50 of the animal. The animal integument portion 50 represents a portion or partial area of the animal's integument, such as the animal's external surface, skin, husk, hide, shell, or rind. Accordingly, when the device 100 is attached to the animal, the attachment layer 140 interposes the housing 120 and the animal integument portion 50, thereby adhering, bonding, or binding the housing 120 to the animal integument portion 50. The device 100 may include a cover layer covering the attachment layer 140, wherein the cover layer is removable to expose the attachment layer 140 before attaching the device 100 to the animal. The animal integument portion 50 may be at any part of the animal, such as the ear, nose, neck, head, hoof, leg, upper part of a tail, or top of a backbone.

As shown in FIG. 1A to FIG. 1C, the animal integument portion 50 may be a skin portion of the animal. The animal integument portion 50 includes three layers—outermost epidermis layer 50a, middle dermis layer 50b, and innermost hypodermis or subcutaneous layer 50c. The epidermis layer 50a, which is made up of epithelial cells and does not contain blood vessels, is mainly functional for protection, absorption of nutrients, and homeostasis. The dermis layer 50b is composed of dense irregular connective tissue and areolar connective tissue. The dermis layer 50b serves to give elasticity to the integument portion 50, allowing for stretching and flexibility. The dermis layer 50b may have hair follicles that regulates hair growth out of the animal integument portion 50, as well as the ends of some vessels including blood and lymphatic vessels. The subcutaneous layer 50c is made of fatty tissue and more vessels, including blood and lymphatic vessels, than the dermis layer 50b. Notably, when the device 100 is attached to the animal integument portion 50, the attachment layer 140 adheres the housing 120 to the epidermis layer 50a and the sensor unit 160 measures physiological signals from the underlying dermis and subcutaneous layers 50bc.

The sensor unit 160 includes a set of physiological sensors 162 for measuring physiological signals from the animal integument portion 50. The sensor unit 160 is engageable with the channel 122, particularly by mating elements between the sensor unit 160 and the channel 122, for axial displacement within the channel 122. When the device 100, specifically the housing 120 thereof, is attached to the animal integument portion 50, the sensor unit 160 is axially displaceable within the channel 122 for adjusting contact with the animal integument portion 50 for measuring the physiological signals. Specifically, when the device 100 is attached to the animal and the sensor unit 160 is engaged with the channel 122, the sensor unit 160 is axially displaceable along the channel 122 towards or away from the animal integument portion 50, thus adjusting the pressure exerted by the sensor unit 160 on the animal integument portion 50. More intimately, the channel 122 provides intricate adjustments for the sensor unit 160 to adjust its contact with the animal integument portion 50.

For example, if the animal integument portion 50 is thick, such as the skin having a thick subcutaneous layer 50c, the sensor unit 160 may need to be displaced further towards and closer to the animal integument portion 50 so as to depress the animal integument portion 50 and strengthen the contact with the animal integument portion 50, thereby increasing the pressure exerted on the animal integument portion 50. This allows the sensor unit 160 to be positioned closer to the dermis and subcutaneous layers 50bc for measuring the physiological signals from the vessels in these layers 50bc.

It will be appreciated that the attachment layer 140 does not cover the channel 122 so that it may be possible for the sensor unit 160 to axially displace past the attachment layer 140. The physiological sensors 162 are arranged on the sensor unit 160 such that the physiological sensors 162 face the animal integument portion 50 when the device 100 is attached to the animal integument portion 50.

Axial displacement of the sensor unit 160 advantageously allows the sensor unit 160 to adjust its contact with the animal integument portion 50 when the device 100, specifically the housing 120, is attached to the animal integument portion 50. For example, when the device 100 is attached to the animal integument portion 50 which may have a curved or contoured profile, the sensor unit 160 may not be properly positioned for good contact with the animal integument portion 50 for the physiological sensors 162 to measure the physiological signals. As shown in FIG. 1A and FIG. 1B, the sensor unit 160 may be too far from the animal integument portion 50 or too loosely contacting the animal integument portion 50 for the physiological signals to be measured accurately. However, it will be appreciated that the sensor unit 160 may be modified with suitable physiological sensors 162 to measure physiological signals without having the physiological sensors 162 being in physical contact with the animal integument portion 50. For example, such physiological sensors 162 may be positioned a small distance, e.g. 1 micron, away from the epidermis layer 50a of the skin.

Conversely, if the sensor unit 160 is adjusted with excessively strong contact with the animal integument portion 50, excessive pressure may be exerted on the animal integument portion 50. Excessively pressuring the animal integument portion 50 may constrict vessels in the animal integument portion 50, particularly in the dermis and subcutaneous layers 50bc, which would cause discomfort to the animal and possibly compromise the physiological signals. Axial displacement of the sensor unit 160 thus allows the sensor unit 160 to be properly contacting the animal integument portion 50, exerting an optimal pressure on the animal integument portion 50, such as shown in FIG. 1C, for optimal measurement of the physiological signals while balancing with any discomfort caused to the animal.

The housing 120 includes a first mating element 124 disposed on the periphery of the channel 122. The sensor unit 160 includes a second mating element 164 disposed on the periphery of the sensor unit 160. The first mating element 124 and second mating element 164 are mutually matingly engageable to thereby engage the sensor unit 160 with the channel 122. The mating elements 124/164 further guide the penetration and interaction between the sensor unit 160 and the channel 122. Additionally, the engagement between the sensor unit 160 and the channel 122 may be water resistant to prevent water from seeping through the engaged elements 124/164 that may compromise physiological signals. The sensor unit 160 may include various sealing elements for preventing liquid or water ingress/seepage into the sensor unit 160.

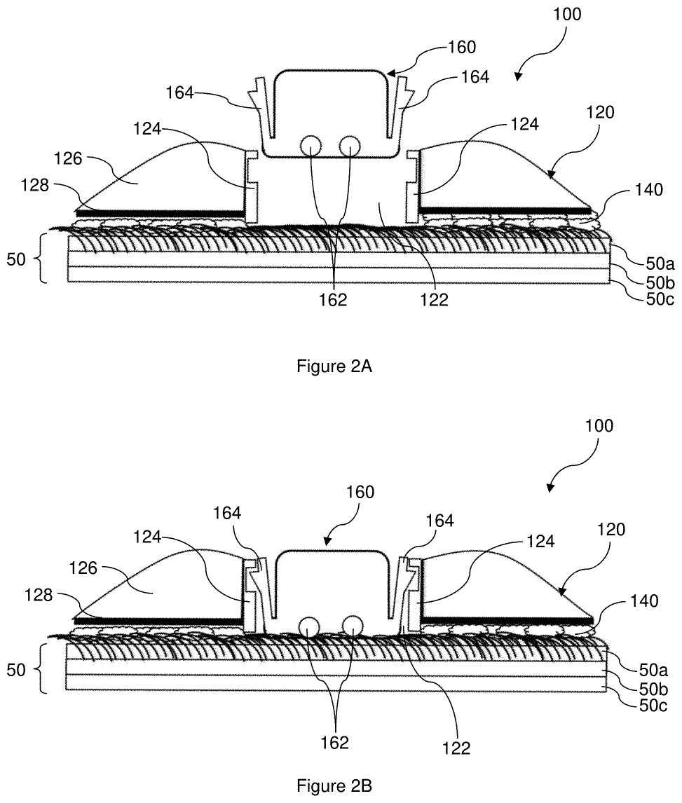

In some embodiments as shown in FIG. 1A to FIG. 1C, the sensor unit 160 is engageable with the channel 122 by a screw mechanism. Specifically, the first mating element 124 and second mating element 164 include matingly engageable screw threads that enable the sensor unit 160 to be axially screwed along the channel 122 towards or away from the animal integument portion 50, stopping at the desired position for optimal measurement of the physiological signals. The screw threads may extend partially or completely across the axial lengths of the sensor unit 160 and the channel 122. In some embodiments as shown in FIG. 2A and FIG. 2B, the sensor unit 160 is engageable with the channel 122 by a clip mechanism. For example, the first mating element 124 includes a fixed clipping element and the second mating element 164 includes a flexible clipping element, wherein the flexible clipping element is engageable with the fixed clipping element to clip and position the sensor unit 160 to the channel 122 at a predefined clip level. The fixed clipping element may be disposed around an internal diameter of the first mating element 124. The flexible clipping element may be disposed at and surrounding at specific positions along the exterior surface portion of the second mating element 164. Additionally, the first mating element 124 may include a plurality of fixed clipping elements such that the flexible clipping element can be clipped to any of the fixed clipping elements, thereby allowing the sensor unit 160 to be clipped to the channel 122 at a plurality of predefined clip levels along the channel 122. The predefined clip levels may differ depending on the type of animals, such as by their species and gender, and may be predetermined based on prior research data on the animals. For example, animals with thicker skin or hide may require the physiological sensors 160 to be positioned much closer to the dermis and subcutaneous layers 50bc of the skin, possibly even depressing against the skin, so that physiological signals can be measured from the blood vessels in these layers 50bc.

In many embodiments, the housing 120 is formed of a resilient material. Specifically, the housing 120 includes a housing body 126 formed of the resilient material, such as silicone or rubber. The housing 120 may be formed with its peripheral regions sloping or tapering downwards, such that when the device 100 forms a streamline profile when attached to the animal integument portion 50, reducing risk of accidental detachment by the animal.

The attachment layer 140 is disposed on a base 128 of the housing body 126. The resilient material of the housing 120 allows the housing 120 to be flexed or contoured to a profile of the animal integument portion 50, thereby allowing the device 100 to be attached at various integument portions 50 of the animal, especially where these animal integument portions 50 have curved or contoured profiles. The housing 120 may be formed by a moulding process using the resilient material, as will be readily understood by the skilled person. In one embodiment, the housing 120 is moulded with the first mating element 124 as an integrated body. In another embodiment, the housing 120 is moulded and the first mating element 124 is coupled to the housing 120.

The channel 122 and the sensor unit 160 are dimensioned such that there is a snug or tight fit when the sensor unit 160 is engaged with the channel 122. In some embodiments, the channel 122 has an internal dimension, e.g. an internal diameter, and the sensor unit 160 has an external dimension, e.g. an external diameter, wherein the external dimension of the sensor unit 160 is slightly larger than the internal dimension of the channel 122, such as 1.1 times larger. Due to the difference in dimensions, the sensor unit 160 must be forced into the channel 122 for engagement therebetween. The engagement of the sensor unit 160 with the channel 122 creates axial forces along the displacement axis of the sensor unit 160, as well as lateral forces between the sensor unit 160 and the housing 120. The sensor unit 160 may comprise a sensor unit holder or gripping portion 166 disposed on a suitable position for ease of displacing the sensor unit 160 along the channel 122 to adjust contact with the animal integument portion 50. The gripping portion 166 may further assist the sensor unit 160 to exert torque along the channel 122.

In an exemplary use case, the device 100 is attached to the animal integument portion 50 and the sensor unit 160 has good contact with the animal integument portion 50 to measure physiological signals. After some time, hair or follicles may grow on the animal integument portion 50 which may destabilize the attachment of the device 100 to the animal integument portion 50. The destabilization affects contact between the sensor unit 160 and the animal integument portion 50, possibly compromising the physiological signals being measured and acquired. In order to re-stabilize the device 100 and re-establish good contact for acquisition of physiological signals from the animal, a user may need to axially displace the sensor unit 160 along the channel 122 outwardly or inwardly to loosen or tighten, respectively, the contact between the sensor unit 160 and the animal integument portion 50. This may be achieved through unscrewing or screwing of the sensor unit 160, respectively. The user may also shave off the excess hair or follicle growth to improve and achieve optimal contact between the sensor unit 160 and the animal integument portion 50.

The creation of the axial and lateral forces can be described as follows. As the sensor unit 160 is axially displaced along the channel 122 towards the animal integument portion 50, the axial forces are created as the sensor unit 160 is forced through the channel 122. Additionally, as the sensor unit 160 is larger than the channel 122, parts of the channel 122 expands, while the sensor unit 160 is going through the channel 122, and contracts subsequently, thereby creating the lateral forces. The combination of the axial and lateral forces induces greater frictional forces between the attachment layer 140 and the animal integument portion 50, thus strengthening the attachment of the device 100 to the animal integument portion 50.

In some embodiments, the mating elements 124/164 include matingly engageable screw threads and the sensor unit 160 may be forced into the channel 122 by applying sufficient torque or rotational force to screw the sensor unit 160 into the channel 122 after the device 100 is attached to the animal integument portion 50. Forcing of the sensor unit 160 into the channel 122 creates the axial forces and the resilient material of the housing 120 may facilitate said forcing as the housing 120 is deformable to accommodate the sensor unit 160 that is slightly larger than the channel 122. The housing 120 thus acts like a spring biasing element that creates the lateral forces between the sensor unit 160 and the housing 120, specifically between the periphery of the sensor unit 160 and the periphery of the channel 122. The combination of the axial and lateral forces may be sufficiently large to curve the housing 120 such that it concaves or collapses inwards towards the animal integument portion 50, allowing the housing 120 to be deformed to the curved or contoured profile of the animal integument portion 50.

In some embodiments, the sensor unit 160 is axially displaceable along a single vector. Upon engagement with the channel 122, the mating elements 124/164 allow the sensor unit 160 to be displaced axially along the channel 122 towards the animal integument portion 50 only, thus preventing removal of the sensor unit 160 from the device 100. For example, the mating elements 124/164 may include a rigid clip mechanism, such as the one used in cable tie or zip tie fasteners, which allow the sensor unit 160 to be displaced in one direction only.

In some embodiments, the sensor unit 160 is disengageable from the channel 122 for removal of the sensor unit 160 from the device 100. For example, the mating elements 124/164 may include a clip mechanism wherein the second mating element 164 includes a flexible clipping element that allow the sensor unit 160 to be unclipped for removal thereof. The mating elements 124/164 may alternatively include a screw mechanism that allow the sensor unit 160 to be screwed inwards and outwards by changing the rotational direction of the sensor unit 160. Removal of the sensor unit 160 allows for replacement thereof, such as if the sensor unit 160 is damaged, so that a new sensor unit 160 or replacement sensor unit 160 can be installed or introduced into the channel 122 of the housing 120 that is still attached to the animal integument portion 50. Ease of replacement of the sensor unit 160 allows for continuous measurement of the physiological signals and monitoring of the physiological conditions. A damaged sensor unit 160 can thus be easily removed for repairs and maintenance. An undamaged sensor unit 160 may also be removed for extraction of physiological data stored thereon and/or for charging.

The device 100 may be attached to any integument portion 50 of the animal, but particularly where the animal integument portion 50 has a good number of vessels for measuring the physiological signals. For example, the animal integument portion 50 is at a lymphatic vessel site of the animal so that the physiological sensors 162 are able to measure the physiological signals from the lymphatic vessels. As the animal is mobile, there is a tendency that the device 100 will slip off from the animal integument portion 50. The attachment layer 140 is thus provided to mitigate risk of slippage.

The attachment layer 140 may include one or more of a bonding agent or adhesive, a touch fastener, and a stub surface. Non-limiting examples of the adhesive include adhesive glue, pliable glue, and heat glue. The adhesive may be biocompatible, such as one containing hydrocolloid. The touch fastener is also known as a hook-and-loop fastener and one way of using this fastener is to attach one of the hook or loop portion to the animal integument portion 50 and attach the other of the hook and loop portion to the housing 120. Attaching of the hook portion and loop portion to the animal skin integument 50 and housing 120 may be by way of an adhesive, e.g. glue. The hook-and-loop fastener may further interact with hairs, follicles, or furs of the integument portion. The stub surface includes a set of stubs for increasing frictional forces between the attachment layer 140 and the animal integument portion 50, thus strengthening the attachment of the device 100 to the animal integument portion 50.

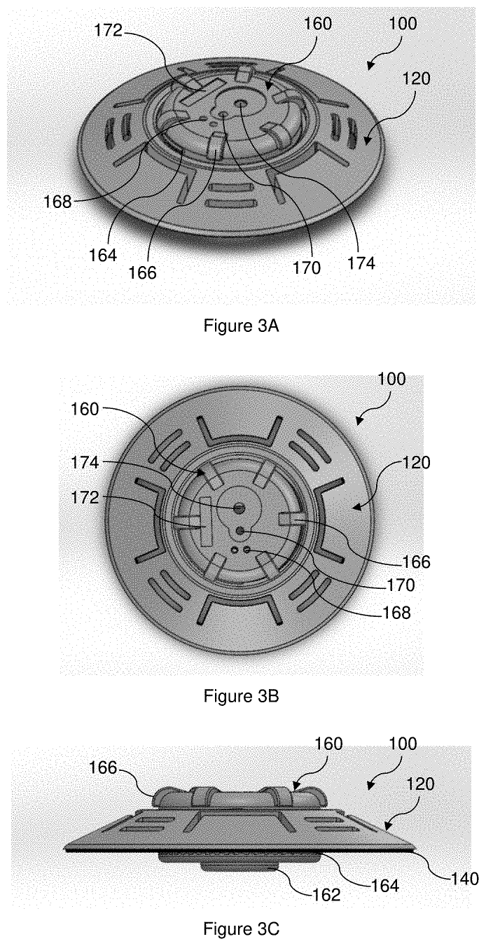

In some embodiments with reference to FIG. 3A to FIG. 3C, the device 100 has a substantially circular shape. Specifically, the housing 120, attachment layer 140, and sensor unit 160 have similar circular shapes. The channel 122 is positioned at a central region of the housing 120 and has a similar circular shape for holding the sensor unit 160. As the shapes are substantially circular, the mating elements 124/164 may include a screw mechanism. The sensor unit 160 may further include a gripping portion 166 for the user to hold when screwing the sensor unit 160 into or out of the channel 122.

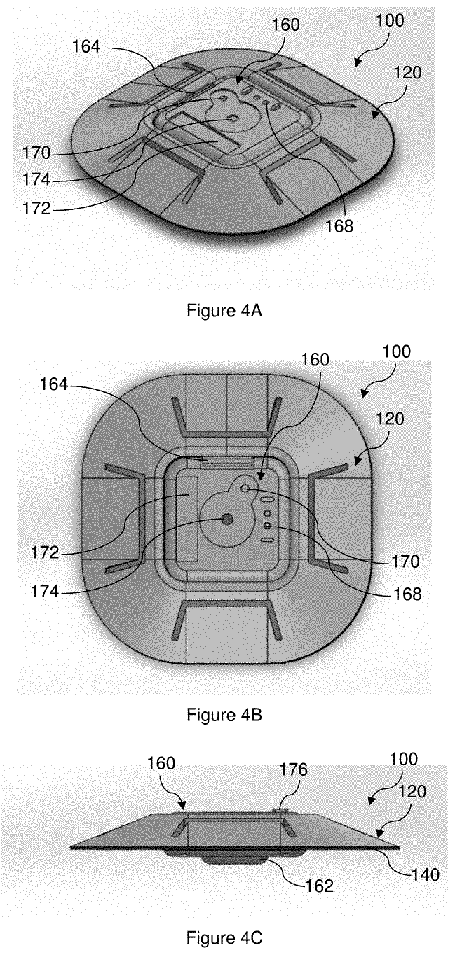

In some embodiments with reference to FIG. 4A to FIG. 4C, the device 100 has a substantially square shape, preferably with rounded or chamfered corners to reduce risk of injury to the animal. Specifically, the housing 120, attachment layer 140, and sensor unit 160 have similar square shapes. The channel 122 is positioned at a central region of the housing 120 and has a similar square shape for holding the sensor unit 160. As the shapes are not circular, the mating elements 124/164 cannot include a screw mechanism, but may instead include a clip mechanism. It will be appreciated that the device 100 may be of various other shapes, such as but not limited to rectangular and elliptical. Compared to the circular physiological device 100 with the screw mechanism, the square physiological device 100 with the clip mechanism is less bulky and has a substantially flatter appearance from its side view. The flatter profile of the device 100 reduces the risk of the device 100 being detached from the animal integument portion 50 due to actions of the animal.

In some embodiments, the housing 120, channel 122, attachment layer 140, and sensor unit 160 may have different shapes. For example, the housing 120 may have a square shape while the channel 122 and sensor unit 160 may have circular shapes. Alternatively, the housing 120 may have a circular shape while the channel 122 and sensor unit 160 may have square shapes. The attachment layer 140 may not be of the same shape as the housing 120. For example, the attachment layer 140 may constitute discrete portions distributed across the surface of the housing 120 for attachment to the animal integument portion 50.

The physiological sensors 162 of the sensor unit 160 are configured to measure the physiological signals of the animal. The physiological signals include one or more of, but are not limited to, heart rate, blood pressure, photoplethysmogram (PPG) signals, and body temperature. The physiological sensors 162 may include one or more photodiode sensors or photodetectors for measuring PPG signals from the vessels at the animal integument portion 50, specifically in the dermis layer 50b and subcutaneous layer 50c. Thus, the physiological sensors 162 should be in good contact with the animal integument portion 50 to be able to optimally measure the PPG signals from the underlying vessels. The physiological sensors 162 may include one or more temperature sensors for measuring body temperature at the animal integument portion 50. It will be appreciated that the physiological sensors 162 may include one or more different types to be used in combination with each other to measure various types of physiological signals from the animal integument portion 50.

The sensor unit 160 may further include a set of illumination elements such as light-emitting diodes (LEDs) to complement the photodiode sensors. Particularly, the animal integument portion 50 is illuminated by the illumination elements and the photodiode sensors measure changes in light absorption to thereby determine detect blood volume changes in the microvascular bed of living tissue in the animal integument portion 50. The illumination elements are configured to emit visible light of any wavelength, such as red light, white light, green light, or lime green light. The illumination elements may be configurable to emit any combination of light as desired. Depending on the integument condition of the animal, the illumination elements may be configured to emit different colours to optimize the physiological signals measured from the animal integument portion 50.

The sensor unit 160 may include an infrared element for emitting infrared radiation. The infrared element may be configured to be activated together with or independent of the illumination elements. The light and/or infrared radiation is emitted to facilitate measurement of the physiological signals by the physiological sensors 162. Like the physiological sensors 162, the illumination elements and infrared element are arranged to face the animal integument portion 50 when the device 100 is attached to the animal integument portion 50. Additionally, the illumination elements are arranged so that the emitted light do not travel directly to the photodiode sensors, as the photodiode sensors are configured to measure changes in light absorption based on reflected light from the animal integument portion 50.

The sensor unit 160 includes an electronic module, such as a printed circuit board, for processing the physiological signals measured by the physiological sensors 162. The sensor unit 160 further includes a power source for powering the sensor unit 160, including the physiological sensors 162 and illumination elements. The power source may include a set of battery cells electrically connected to the physiological sensors 162 and the electronic module. The arrangement and number of battery cells may be predetermined based on the power requirements of the sensor unit 160. For example, the device 100 may be required to measure physiological signals and monitor physiological conditions of the animal for at least 2 weeks, and the battery cells would be configured accordingly, as will be readily understood by the skilled person.

The battery cells may be chargeable, such as lithium-ion polymer batteries. The sensor unit 160 may include a set of electrical or charge contacts 168 connectable to an electrical supply for charging the battery cells. In one embodiment, the battery cells may be charged by magnetic charging means. The housing 120 may include a charging holder 176 for holding or supporting the device 100 on a charging device whereto the device 100 is connected for charging. In one embodiment, the sensor unit 160 may further include a set of solar cells configured to receive solar power from the sun and to charge the battery cells with the solar power. The solar cells are arranged on the sensor unit 160 such that they are facing the sun when the device 100 is attached to the animal. Inclusion of the solar cells allows for charging of the battery cells in daylight so that the sensor unit 160 can operate through the day and night, ensuring that the device 100 remains functional on the animal even after long periods of use.

The sensor unit 160 may include a set of visual indicators 170, such as LEDs, to generate visual alerts based on the physiological signals. The sensor unit 160 may include a labelling area 172 for placing an identifier of the device 100. This identifier may also be used for identification of the animal whereon the device 100 is attached. The sensor unit 160 may include a set of actuation elements 174 for performing various functions of the sensor unit 160. For example, an actuation element 174 may be activated for communication of the measured physiological signals, while another actuation element 174 may be activated to reset the sensor unit 160, such as in event of failure to measure physiological signals. In one embodiment, the actuation elements 174 are in the form of physical buttons. In another embodiment, the actuation elements 174 are provided via a user interface on a touch screen display of the sensor unit 160.

One or more physiological devices 100 may be attached to an animal at respective integument portions 50 of the animal for measuring physiological signals therefrom. For each device 100, the attachment layer 140 of the device 100 is first attached or adhered to the animal integument portion 50. The sensor unit 160 is engaged with the channel 122 in the housing 120 for axial displacement within the channel 122. When the device 100 is attached to the animal integument portion 50, the sensor unit 160 is axially displaceable within the channel 122 for adjusting contact of the sensor unit 160 with the animal integument portion 50, allowing the sensor unit 160 to be properly positioned for good contact with the animal integument portion 50 for optimal measurement of the physiological signals. The physiological signals cannot be measured accurately if the contact between the sensor unit 160 and the animal integument portion, and consequently the pressure exerted on the animal integument portion 50, is not optimal. Particularly, the physiological signals may be compromised if the physiological sensors 162 is overly forcing against and excessively depressing the animal integument portion 50, which would constrict vessels at the animal integument portion 50, as well as cause discomfort and induce unnecessary stress to the animal.

Further, the sensor unit 160 is forced into the channel 122 for engagement therebetween, thus creating axial and lateral forces as described above. The combination of the axial and lateral forces induces greater frictional forces between the attachment layer 140 and the animal integument portion 50, thus strengthening the attachment of the device 100 to the animal integument portion 50 and reducing risk of accidental detachment of the device 100 from the animal integument portion 50. The risk of accidental detachment exists because animals 60 are inquisitive by nature and will attempt to dislodge any foreign objects that are placed on their bodies. There is also a tendency that the animals 60 will nibble on the foreign objects, thus potentially dislodging or moving the foreign objects. In adverse conditions, the animals 60 may choke on these foreign objects that may eventually cause death. Further, when the animal moves between a standing position and a resting position, it is likely that the animal will brush against another structure, e.g. a fence, or another animal that may knock or move the foreign object.

As it can be difficult to handle the animal, the small and robust design of the physiological device 100 makes it easy and less laborious to be deployed and attached to the animal, including in open fields and in holding yards. The strong attachment between the device 100 and animal integument portion 50 due to the attachment layer 140 and increased frictional forces reduces risk of accidental detachment of the device 100 despite attempts by the animal to dislodge it. The shape and size of the device 100 also reduce risk that the animal will accidentally consume the device 100, thus preventing choking on the device. The reduced risk of detachment allows the device 100 to be constantly attached to the animal integument portion 50, thereby maintaining continuous measurement of physiological signals and monitoring of physiological conditions of the animal. Useful knowledge and insights can thus be obtained from the continuous measurements and monitoring. As the device 100 is constantly attached to the animal and the sensor unit 160 can be removed and replaced, it would not be necessary to periodically replace the device 100 in its entirety, which can cause unnecessary discomfort and stress to the animal and which also usually requires trained professionals to fixate anything onto the animal to avoid agitating it.

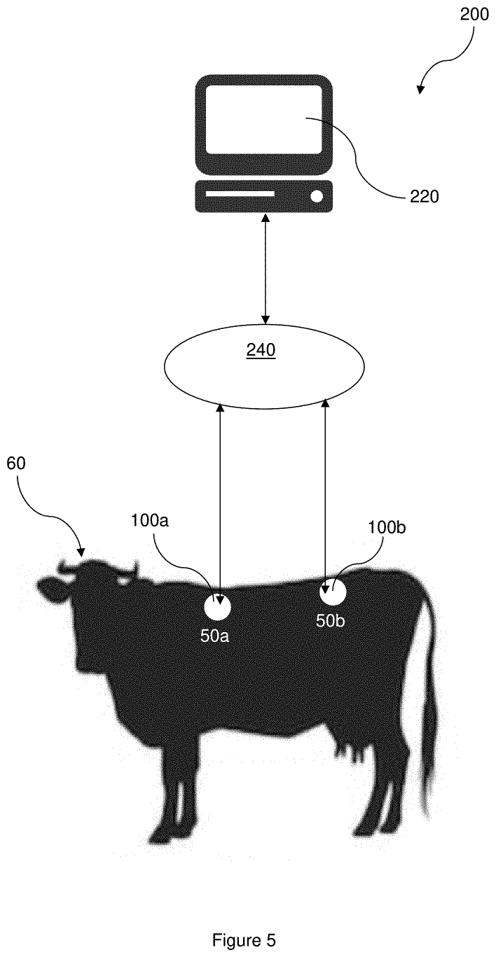

In various embodiments of the present disclosure with reference to FIG. 5, there is a system 200 for monitoring physiological conditions of an animal 60. The system includes a set of physiological devices 100 attachable to the animal 60 and further includes an electronic device 220 communicative with the physiological devices 100 for processing the physiological signals to thereby monitor the physiological conditions of the animal 60. It will be appreciated that the system 200 is capable of monitoring the physiological conditions of more than one or numerous animals 60, such as cattle in a holding yard or farm, wherein each animal 60 has one or more physiological devices 100 attached thereto. The electronic device 220 may be configured for activating and deactivating each physiological device 100, and for selectively extracting the measured physiological signals.

The electronic device 220 may be a mobile device, such as mobile phone, smartphone, personal digital assistant (PDA), tablet, laptop, or computer. Alternatively, the electronic device 220 is a remote server that is a physical or cloud data processing system and includes one or more computers, laptops, mini-computers, mainframe computers, any non-transient and tangible machines that can execute a machine-readable code, cloud-based servers, distributed server networks, and a network of computer systems. The electronic device 220 includes a processor, a memory, and various other modules or components. The modules and components thereof are configured for performing various operations or steps and are configured as part of the processor. Such operations or steps are performed in response to non-transitory instructions operative or executed by the processor. The memory is used to store instructions and perhaps data which are read during program execution. The memory may be referred to in some contexts as computer-readable storage media and/or non-transitory computer-readable media. Non-transitory computer-readable media include all computer-readable media, with the sole exception being a transitory propagating signal per se.

The electronic device 220 is communicative with the physiological devices 100 across a communication network 240, such as by wireless communication protocols, as will be readily understood by the skilled person. In one example, the communication network 240 may be a short range, such as radio frequency identification (RFID), Wi-Fi, Bluetooth Low Energy (BLE), or Near Field Communication (NFC). In another example, the communication network 240 may be long range, such as Local Area Network (LAN), Wireless Area Network (WAN), telecommunication network, cellular network, satellite network, or LoRa WAN (Long Range WAN).

In one embodiment, the physiological signals measured by each device 100 are stored on a database residing within the device 100. The measured physiological signals may be communicated from the sensor unit 160 to the electronic device 220 in response to actuation of an actuation element 174 or in response to remote activation by the electronic device 220. Alternatively, the sensor unit 160 is removed from the device 100 and connected to the electronic device 220 to download the measured physiological signals. In another embodiment, the measured physiological signals are streamed on-the-fly to the electronic device 220 via the communication network 240.

The system 200 may be implemented in holding yard having a number of animals 60, e.g. cattle, each having one or more physiological devices 100 attached thereto. The electronic device 220 may be located in the premises of the holding yard to receive the physiological signals from the devices 100. As shown in FIG. 5, for an exemplary animal 60 such as a cow, there are two physiological devices 100 attached to respective integument portions 50 of the animal 60 for measuring physiological signals from the animal 60. Specifically, a first device 100a is attached to a first integument portion 50a at the dorsal or shoulder area of the animal 60, and a second device 100b is attached to a second integument portion 50b at the lumbar area of the animal 60.

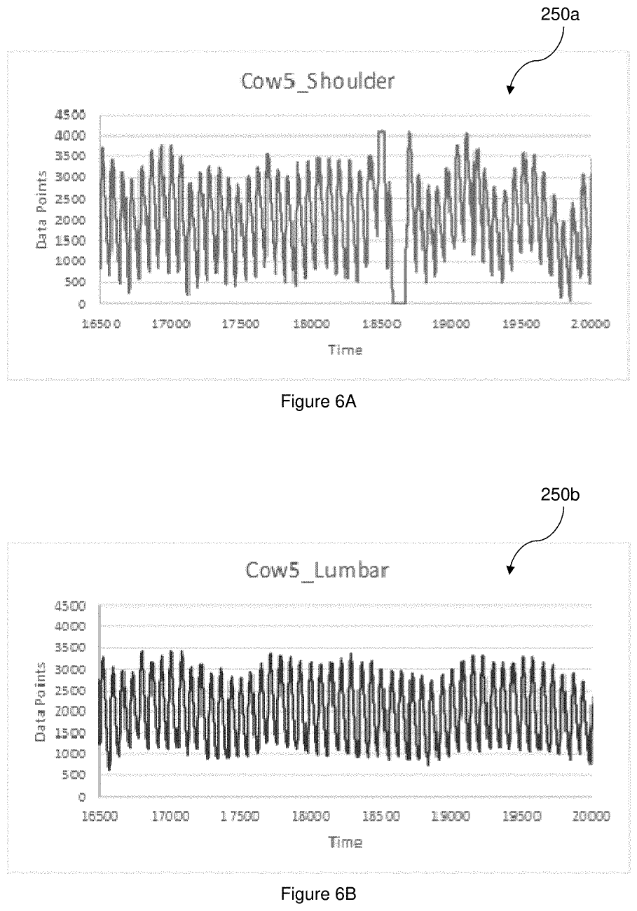

The physiological signals measured from the first and second integument portions 50ab are communicated to the electronic device 220 via the communication network 240 for processing to thereby monitor the physiological conditions of the animal 60. As shown in FIG. 6A, the chart 250a illustrates an example of the measured physiological signals against time at the first integument portion 50a. As shown in FIG. 6B, the chart 250b illustrates an example of the measured physiological signals against time at the second integument portion 50b.

The physiological conditions of the animal 60 are monitored by processing the measured physiological signals, such as by way of an algorithm used to monitor the stress of the animal 60. In one example, the physiological signals may include heart rate and the heart rate signals may be processed to determine physiological data such as the mean heart rate, standard deviation of the heart rate, pulse shape feature, and the like. In another example, the physiological signals may include body temperature of the animal 60 which can be processed to assess how environmental, weather, and/or climate changes impact the animal's body temperature over time. The behavioral status of the animal 60, particularly the stress condition, can be used to indicate the general state of health of the animal 60, such as prediction of milk quality, milk fever, beef quality, diseases or calving occurrence condition is good or acceptable, and thus indicate a wellness pattern of the animal 60. The physiological data can thus provide better knowledge about the physiological conditions and health of the animal 60, including its present state of mind.

In the foregoing detailed description, embodiments of the present disclosure in relation to an animal physiological device are described with reference to the provided figures. The description of the various embodiments herein is not intended to call out or be limited only to specific or particular representations of the present disclosure, but merely to illustrate non-limiting examples of the present disclosure. The present disclosure serves to address at least one of the mentioned problems and issues associated with the prior art. Although only some embodiments of the present disclosure are disclosed herein, it will be apparent to a person having ordinary skill in the art in view of this disclosure that a variety of changes and/or modifications can be made to the disclosed embodiments without departing from the scope of the present disclosure. Therefore, the scope of the disclosure as well as the scope of the following claims is not limited to embodiments described herein.

1. A physiological device for an animal, the device comprising:
a housing comprising a channel therethrough;
an attachment layer disposed on the housing for attaching the device to an integument portion of the animal; and
a sensor unit comprising a set of physiological sensors for measuring physiological signals from the animal integument portion, the sensor unit engageable with the channel for axial displacement within the channel,
wherein when the device is attached to the animal integument portion, the sensor unit is axially displaceable within the channel for adjusting contact with the animal integument portion for measuring the physiological signals.

2. The device according to claim 1, wherein the sensor unit is engageable with the channel by a screw mechanism.

3. The device according to claim 1, wherein the sensor unit is engageable with the channel by a clip mechanism.

4. The device according to claim 3, wherein the clip mechanism is configured such that the sensor unit is clippable to the channel at a plurality of predefined clip levels along the channel.

5. The device according to claim 1, wherein the engagement between the sensor unit and the channel is water resistant.

6. The device according to claim 1, wherein the sensor unit is axially displaceable along a single vector.

7. The device according to claim 1, wherein the sensor unit is disengageable from the channel for removal of the sensor unit.

8. The device according to claim 1, wherein the set of physiological sensors comprises one or more photodiode sensors for measuring photoplethysmogram (PPG) signals from the animal integument portion.

9. The device according to claim 1, wherein the housing is formed of a resilient material.

10. The device according to claim 1, wherein the housing is contourable to a profile of the animal integument portion.

11. The device according to claim 1, wherein the attachment layer comprises one or more of an adhesive, a touch fastener, and a stub surface.

12. The device according to claim 1, further comprising a cover layer covering the attachment layer, wherein the cover layer is removable before attaching the device to the animal.

13. A system for monitoring physiological conditions of an animal, the system comprising:
a set of physiological devices attachable to the animal, each device comprising:
a housing comprising channel therethrough;
an attachment layer disposed on the housing for attaching the device to an integument portion of the respective animal; and
a sensor unit comprising a set of physiological sensors for measuring physiological signals from the animal integument portion, the sensor unit engageable with the channel for axial displacement within the channel, wherein when the device is attached to the animal integument portion, the sensor unit is axially displaceable within the channel for adjusting contact with the animal integument portion for measuring the physiological signals; and
an electronic device communicative with the physiological devices for processing the physiological signals to thereby monitor the physiological conditions of the animals.

14. The system according to claim 13, wherein for each device, the sensor unit is engageable with the channel by a screw mechanism.

15. The system according to claim 13, wherein for each device, the sensor unit is engageable with the channel by a clip mechanism.

16. The system according to claim 13, wherein for each device, the sensor unit is disengageable from the channel for removal of the sensor unit.

17. The system according to claim 13, wherein for each device, the housing is formed of a resilient material.

18. The system according to claim 13, wherein for each device, the housing is contourable to a profile of the respective animal integument portion.

19. The system according to claim 13, wherein for each device, the attachment layer comprises one or more of an adhesive, a touch fastener, and a stub surface.

20. The system according to claim 13, wherein the electronic device is configured for activating and deactivating each physiological device.
